(19) United States
(12) Patent Application Publication
(10) Pub. No.: US 2005/0274378 A1
(43) Pub. Date: Dec. 15, 2005

(54) Medicament dispenser

(76) Inventors: Stanley George Bonney, Ware (GB); Michael Birsha Davies, Ware (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/523,066

(22) PCT Filed: Jul. 23, 2003
(86) PCT No.: PCT/EP03/08152

§ 371 Date: Jan. 24, 2005

(30) Foreign Application Priority Data
Jul. 25, 2002 (GB) 0217198.1

(51) Int. Cl. A61M 11/00; A61M 15/00

(52) U.S. Cl. 128/200.23; 128/203.15; 128/203.12

(57) ABSTRACT

A unitary dispensing device for use in the delivery of a first medicament and at least one further medicament. The device comprises a first medicament dispenser for the delivery of the first medicament and at least one further medicament dispenser for the delivery of the at least one further medicament. The first medicament dispenser and the at least one further medicament dispenser enable the first and the at least one further medicament to be kept separate until the point of delivery. The first medicament dispenser is different in type to the at least one further medicament dispenser.

### TECHNICAL FIELD

The present invention relates to a medicament dispenser for dispensing medicament combination products. The invention particularly relates to a device for use in dispensing medicament in mixed product form.

### BACKGROUND TO THE INVENTION

The use of inhalation devices in the administration of medicaments, for example in bronchodilation therapy is well known. Such devices generally comprise a body or housing within which a medicament carrier is located. Known inhalation devices include those in which the medicament carrier is a blister strip containing a number of discrete doses of powdered medicament. Such devices usually contain a mechanism of accessing these doses, usually comprising either piercing means or means to peel a lid sheet away from a base sheet. The powdered medicament can then be accessed and inhaled. Other known devices include those in which the medicament is delivered in aerosol form, including the well-known metered dose inhaler (MDI) delivery devices. Liquid-based inhaler devices are also known.

Therapies involving combinations of different and complementary active medicaments are known. These can be administered either as distinct combination (i.e. multi-active) medicament products, which comprise a defined mixture of each component medicament, or as groups of single active medicament products, which are designed to be taken in combination or sequentially. Whilst combination products offer added convenience for the patient, certain medicament actives are difficult to formulate as distinct combination products. For example, the actives may interact chemically with each other in an undesirable way when formulated together.

It is thus, desirable in certain circumstances, to have a medicament dispenser that separately (i.e. in isolated fashion) contains each active component of a combination product, but which enables the delivery of a combined dose in response to a minimum number of patient actions. In particular, it is desirable that each active component of the combined dose is delivered to the patient in a single, combined dose in response to a single patient dosing action. For example, it is desirable that a combined product for inhalation be delivered in response to a single patient actuation of an inhaler, even where the active components of that combined product are separately stored within the inhaler device. Various types of inhaler are known in the art including propellant-based, metered dose inhalers (MDIs); dry powder inhalers (DPIs); and liquid-based, spray inhalers (LSIs).

The Applicants have now observed that particular medicaments can be more suited to delivery to by particular types of inhaler device. For example, one particular medicament may be more suitable for delivery by an MDI device, whereas another may be more suitable for delivery by a DPI device. That suitability may for example, be driven by ease of formulation of the medicament for that particular inhaler device or by the delivery and pharmaceutical performance characteristics obtainable when the particular inhaler device is employed.

The Applicants have also now realised that this preferential suitability can potentially either restrict development choice, or lead to compromise in pharmaceutical performance, when developing an inhaler device for any particular combination product. The problem is encountered where one component medicament of a combination product is suited to delivery by one particular type of inhaler device, whereas the other component medicament of that combination product is suited to delivery by a different type of inhaler device.

Whilst the above problem may be addressed by separate formulation of the components and sequential delivery thereof by separate and different types of inhaler, this is inconvenient for the patient and can lead to confusion in situations where for example, the different inhalers contain different numbers of doses of medicament.

The Applicants therefore now provide, in solution to the above problem, a single inhaler device, which comprises in combination different inhaler types (e.g. combined DPI and MDI; MDI and LSI; or DPI and LSI). The device enables convenient, combined delivery of the components of a combination medicament product to a patient. Suitably, the delivery of the medicament combination occurs on a simultaneous basis and is responsive to a minimum number of patient actions (e.g. single patient actuation or inhalation step).

The Applicants have also realised that using a single inhaler device of the type described above can potentially reduce the complexity, timescale and cost of the development process for a particular drug product because it enables the optimum (e.g. from a development simplicity) delivery vehicle to be selected for each particular medicament component of the combination. The additional development complexity, which is often associated with formulating combination products is thereby effectively avoided.

### SUMMARY OF THE INVENTION

According to one aspect of the invention there is provided a unitary device for use in the delivery of a first medicament and at least one further medicament as a combination medicament product, the device comprising
a first medicament dispenser for the delivery of said first medicament; and
at least one further medicament dispenser for the delivery of said at least one further medicament,
wherein said first medicament dispenser and said at least one further medicament dispenser enable the first and the at least one further medicament to be kept separate until the point of delivery, and the first medicament dispenser is different in type to the at least one further medicament dispenser.

In combination, the first medicament and at least one further medicament comprise a defined combination product. That is to say, that when combined together the distinct active medicament doses released by actuation of the device form a dose of a ‘multi-active’ medicament treatment.

On actuation, the unitary device is designed to deliver a dose portion of the first medicament and a dose portion of each at least one further medicament. The term ‘dose portion’ is employed because in the context of the invention the distinct ‘portions’ are brought together on delivery to form a combination (i.e. multi-active) product dose.

In one particular aspect, the unitary device is designed to receive the first and only one further medicament dispenser. Thus, the device functions as a bi-dispenser device.

In one aspect, the first medicament container and the at least one further medicament container are of a type adapted to be used with a medicament dispenser selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI). The first medicament dispenser and at least one further remain different in type.

In one aspect, the first medicament dispenser is a reservoir dry powder inhaler (RDPI), and the at least one further medicament dispenser is of a type selected from the group consisting of a multi-dose dry powder inhaler (MDPI), a metered dose inhaler (MDI) and a liquid spray inhaler (LSI).

In another aspect, the first medicament dispenser is a multi-dose dry powder inhaler (MDPI), and the at least one further medicament dispenser is of a type selected from the group consisting of a reservoir dry powder inhaler (RDPI), a metered dose inhaler (MDI) and a liquid spray inhaler (LSI).

In another aspect, the first medicament dispenser is a unit dose dry powder inhaler (UDPI), and the at least one further medicament dispenser is of a type selected from the group consisting of a metered dose inhaler (MDI) and a liquid spray inhaler (LSI).

In a further aspect, the first medicament dispenser is a metered dose inhaler (MDI), and the at least one further medicament dispenser is of a type selected from the group consisting of a reservoir dry powder inhaler (RDPI), a multi-dose dry powder inhaler (MDPI) and a liquid spray inhaler (LSI).

In a further aspect, the first medicament dispenser is a liquid spray inhaler (LSI), and the at least one further medicament dispenser is of a type selected from the group consisting of a reservoir dry powder inhaler (RDPI), a multi-dose dry powder inhaler (MDPI) and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By unit dose dry powder inhaler (UDPI) it is meant an inhaler suitable for dispensing medicament in dry powder form, wherein the medicament is comprised within a unit dose container pack containing a single dose (or part thereof) of medicament product. In a preferred aspect, the carrier has a capsule-based pack form.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof of medicament. In a preferred aspect, the carrier has a blister pack form, but it could also, for example, comprise a capsule-based pack form or a carrier onto which medicament has been applied by any suitable process including printing, painting and vacuum occlusion.

In one aspect, the multi-dose pack is a blister pack comprising multiple blisters for containment of medicament in dry powder form. The blisters are typically arranged in regular fashion for ease of release of medicament therefrom.

In one aspect, the multi-dose blister pack comprises plural blisters arranged in generally circular fashion on a disc-form blister pack. In another aspect, the multi-dose blister pack is elongate in form, for example comprising a strip or a tape.

Preferably, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined therebetween and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085.

By liquid spray inhaler (LSI) it is meant a medicament dispenser suitable for dispensing medicament in spray form, wherein the medicament is typically formulated in liquid or solution form and comprised in a liquid container. The container is typically provided with a means of metering to a spray generator, which imparts energy to the liquid or solution, thereby generating a spray for inhalation by the patient. The spray generator, in aspects, comprises a vibrating element (e.g. a mesh) that provides vibrational energy to the formulation, thereby resulting in its aerosolisation. In other aspects, the spray generator comprises a pump mechanism, which either delivers the medicament directly to the patient (as a liquid spray) or which delivers the medicament to an intermediate position at which further energy is supplied thereto to further propel, aerosolise or otherwise direct the medicament dose to the patient.

The medicament device has unitary form, and typically has a housing shaped to receive, and enable the patient actuation of, the first and at least one further medicament dispensers.

In one aspect, the housing integrally comprises an actuator for at least one, preferably all of the medicament dispensers. Suitably, the actuator for each medicament dispenser is coupled, thereby enabling simultaneous delivery of medicament from each dispenser in response to a single patient actuation step.

In another aspect, the housing is shaped to receive medicament dispensers, which are provided with respective actuators. In this case, the actuators have typically been adapted for receipt by the housing. The medicament dispenser and actuator therefor are in one aspect, supplied as independently operable ‘cassette refills’ for the unitary device.

In one aspect, the device is provided with mixing means for ensuring mixing of the delivered medicaments prior to their inhalation by the patient as a ‘mixed’ combination product.

Suitably, the mixing means comprises a mixing chamber including inlets for receiving medicament form each medicament dispenser and an outlet for delivery of ‘mixed’ medicament product to the patient for inhalation (e.g. through a mouthpiece which communicates with the mixing chamber). The ergonomics of the mixing chamber will be arranged to ensure effective mixing of the separate medicament feeds. In aspects, baffles, propellers, venturi and other features for controlling mixing dynamics are provided. The mixing chamber may also be provided with energisation means for energising the mixing process, or alternatively features may be provided to harness the energy provided by a patient's inward breath to enhance the mixing process.

The device may be provided with means for varying the amount of medicament product released from each medicament dispenser. Customized delivery of combination medicament product may therefore be achieved through varying the relative ratios of each individual medicament product delivered as well as by varying the absolute amount of medicament delivered. Variable timing mechanisms are envisaged for achieving such customisation.

In one aspect, the medicament dispenser herein includes a timing control system for controlling the time of release of contents from the first and at least one further medicament container. The timing control system generally communicates with an electronic control system with which it may in aspects, form an integral part.

The timing control system is suitably arranged to vary the relative time of release of each medicament component from its respective medicament container. Each medicament component may therefore be arranged for simultaneous or sequential release, although in general where components are released sequentially the time delay between releases of each separate medicament component is short (e.g. milliseconds) to ensure that a combined product is provided for administration to the patient.

In a further aspect, by varying the time of release, the ratio of quantity of each medicament component released can also be varied, thereby enabling the provision and delivery of ‘tailored’ combined products.

Delivery of the combination product (e.g. after mixing) to the patient is preferably through a single outlet. The outlet is typically positioned to be in communication with the distinct medicament dose portions delivered. The outlet may have any suitable form. In one aspect, it has the form of a mouthpiece and in another it has the form of a nozzle for insertion into the nasal cavity of a patient.

Delivery of the combination product (e.g. after mixing) to the patient is preferably through a single outlet. The outlet is typically positioned to be in communication with the distinct medicament dose portions delivered. The outlet may have any suitable form. In one aspect, it has the form of a mouthpiece and in another, it has the form of a nozzle for insertion into the nasal cavity of a patient.

The outlet is preferably a single outlet, which communicates with the distinct medicament dose portions delivered via a common air channelling means (e.g. formed as an air-pipe or common manifold). The patient may therefore breathe in through a single outlet, and that breath be transferred through the common channelling means to (all of) the released medicament dose portions, thereby enabling their inhalation as a combined product.

In addition to, or as an alternative to, any separate mixing chamber, the outlet and/or channelling means may be shaped to encourage mixing of medicament as a result of the air flow created by inhalation by the patient. For example, baffles or other mechanical aids to mixing may be incorporated. Venturi channelling of the air flow is also envisaged in embodiments. Helical form channels are envisaged.

Any or all mechanical components of the device may be driven by either an electronic or mechanical drive system or combination thereof.

Suitably electronic drive means typically comprise a motor, preferably an electrically-powered motor. The motor may provide linear or rotary drive, but in general, rotary motors are most suitable. The motor may for example, comprise a DC electric motor, a piezoelectric (PZ) motor, an ultrasonic motor, a solenoid motor or a linear motor. Preferably, the electronic drive system comprises a DC motor, a PZ motor or an ultrasonic motor.

The use of ultrasonic motors is particularly preferred since they offer advantages over conventional motors in terms of weight, size, noise, cost and torque generated. Ultrasonic motors are well known in the art and are commercially available (e.g. BMSTU Technological Cooperation Centre Ltd, Moscow, Russia; Shinsel Corporation, Tokyo, Japan).

Ultrasonic motors do not use coils or magnets but comprise a piezo-electric ceramic stator which drives a coupled rotor. The stator generates ultrasonic vibrations which in turn causes rotation of the rotor. While regular DC motors are characterised by high speed and low torque, requiring reduction gearing to increase torque, ultrasonic motors attain low speed and high torque, thus eliminating the need for reduction gearing. Furthermore, these motors are lightweight and compact, lacking coils and magnets, and are noiseless as the ultrasonic frequencies used are not audible to the human ear.

Suitably, the device further comprises actuating means for actuating said electronic drive system. Said actuating means may take the form of a switch, push-button, or lever.

Suitably, the device additionally comprises an electronic data management system. The electronic data management system has input/output capability and comprises a memory for storage of data; a microprocessor for performing operations on said data; and a transmitter for transmitting a signal relating to the data or the outcome of an operation on the data.

Suitably, the electronic data management system is arranged to be responsive to or activated by the voice of a user. Thus, for example the system may be switched on or off in response to a voice command.

The electronic data management system may be integral with the body. Alternatively, the electronic data management system forms part of a base unit which is reversibly associable with the body.

Suitably, the device additionally comprises a data input system for user input of data to the electronic data management system. Preferably, the data input system comprises a man machine interface (MMI) preferably selected from a keypad, voice recognition interface, graphical user interface (GUI) or biometrics interface.

Energy may be conserved by a variety of means to enable the device to operate for longer on a given source of energy, such as a battery. Energy conservation or saving methods have additional advantages in terms of reducing the size requirements of the power source (e.g. battery) and thus the weight and portability of the medicament dispenser.

A variety of energy saving methods is available which generally involve reducing power consumption. One such method is to use a clock or timer circuit to switch the power on and off at regular or predetermined intervals. In another method the system can selectively switch on/off specific electronic devices, such as visual display units or sensors, in order to power these devices only when they are required to perform a particular sequence of events. Thus different electronic devices may be switched on and off at varying intervals and for varying periods under control of the system. The power sequencing system may also respond to a sensor, such as a motion or breath sensor, which is activated on use of the device.

Low power or “micropower” components should be used within the electronics where possible and if a high power device is required for a particular function this should be put into a low power standby mode or switched off when not required. Similar considerations apply in the selection of transducers. Operation at low voltage is desirable since power dissipation generally increases with voltage.

For low power digital applications complementary metal oxide semi-conductor (CMOS) devices are generally preferred and these may be specially selected by screening for low quiescent currents. Clock speeds of processors and other logic circuits should be reduced to the minimum required for computational throughput as power consumption increases with frequency. Supply voltages should also be kept at minimal values consistent with reliable operation because power dissipation in charging internal capacitance's during switching is proportional to the square of the voltage. Where possible, supply voltages should be approximately the same throughout the circuit to prevent current flowing through input protection circuits. Logic inputs should not be left floating and circuits should be arranged so that power consumption is minimised in the most usual logic output state. Slow logic transitions are undesirable because they can result in relatively large class-A currents flowing. Resistors may be incorporated in the power supply to individual devices in order to minimise current in the event of failure.

In some control applications, devices that switch between on and off states are preferred to those that allow analog (e.g. linear) control because less power is dissipated in low resistance on states and low current off states. Where linear components are used (e.g. certain types of voltage regulators) then types with low quiescent currents should be selected. In some circuit configurations it is preferable to use appropriate reactive components (i.e. inductors and capacitors) to reduce power dissipation in resistive components.

Suitably, the system additionally comprises a visual display unit for display of data from the electronic data management system to the user. The display may for example, comprise a screen such as an LED or LCD screen. More preferably the visual display unit is associable with the body of the medicament dispenser.

Suitably, the device additionally comprises a datalink for linking to a local data store to enable communication of data between the local data store and the electronic data management system. The datastore may also comprise data management, data analysis and data communication capability.

The datastore may itself form part of a portable device (e.g. a handheld device) or it may be sized and shaped to be accommodated within the patient's home. The datastore may also comprise a physical storage area for storage of replacement cassettes. The datastore may further comprise a system for refilling medicament from a reservoir of medicament product stored therewithin. The datastore may further comprise an electrical recharging system for recharging any electrical energy store on the medicament dispenser, particularly a battery recharging system.

The datalink may for example enable linking with a docking station, a personal computer, a network computer system or a set-top box by any suitable method including a hard-wired link, an infrared link or any other suitable wireless communications link.

In one aspect, the device includes an electronic dose reminder system. This may be configured to have any suitable form and may be powered by a mains, stored (e.g. battery) or self-regenerating (e.g. solar) energy power source.

The electronic dose reminder system comprises an electronic timer for timing an elapsed time period corresponding to the time since the last actuation of the device; a dose interval memory for storing data relating to a prescribed dose interval time period; and a patient alerter for alerting a user. The alerter activates when the elapsed time period exceeds the prescribed dose interval time period.

The electronic timer progressively times the period since the last actuation of the device (the ‘elapsed time period’). The timer can have any suitable electronic form. The significance of the ‘elapsed time period’ is that in use, it typically corresponds to the time elapsed since the previous dose delivery event.

The timer may be configured to include an automatic re-zeroing feature such that on subsequent actuation of the device the timer count starts again from zero.

The dose interval memory stores data relating to a prescribed dose interval time period. By way of examples, if the medicament is to be taken twice a day at a regular interval, the prescribed dose interval may be set as twelve hours, or for a once daily treatment the value may be set at twenty four hours. In aspects, the system may be configured to allow for ready readjustment of the prescribed dose interval time period, or it may be configured in secure fashion such that any readjustment may be made only by a designated prescriber (e.g. a medical professional or pharmacist). Password and/or other security means may be employed. The prescribed dose interval may be configured to be variable over a particular course of treatment, or alternatively it may be fixed at a set dose interval over the full course of treatment.

The patient alerter is designed to communicate an alert to the user. The alerter activates only when the holding time period exceeds the prescribed dose interval time period. By way of an example, for a once daily treatment with a prescribed dose interval of twenty four hours, the alerter would activate only when the holding time period, as timed by the electronic timer, exceeds twenty four hours since at this point another dose is due to be taken. It may thus, be appreciated that the alerter acts functionally as a reminder to the patient that a dose is due to be taken.

The alerter may in aspects, comprise a visual device, such as a liquid crystal display (LCD) or an array of light-emitting diodes (LEDs), connected to a battery-driven timing device of any convenient kind known to those skilled in the art. The visual device may be configured to display information such as the actual time or the elapsed time from the taking of a previous dosage and may have superimposed thereon additional messages, such as a textual instruction to take a dose of the medicament. Alternatively, the instruction to take the medicament may be conveyed merely by displaying a warning colour or by causing the display to flash or in any other way.

In a further alternative arrangement, no specific time or elapsed time information is displayed, but the alerter merely provides a warning signal that indicates the necessary action to the user.

Depending upon the lifestyle of the user, additional or alternative warnings may be of greater assistance than purely visual warnings. Accordingly, the invention envisages that the alerter may provide audible and/or tactile warnings, such as vibration, instead of (or in addition to) visual warnings.

The alerter may provide a single, one-off alert. More preferably, the alerter is configured to provide the alert over a set period of time (the ‘alerting time period’ or ‘alerting window’). In one aspect, the alerting time period is calculated as a function of (e.g. fraction of) the dose interval time period. For example, for a twice daily treatment with a dose interval time period of twelve hours, the alerting time period may be set as half that period (i.e. six hours). In this case, the alert is then provided for the six hours immediately following the activation of the alert.

The system is typically configured such that the alerting signal cuts off when the user removes the medicament delivery device from the holder to enable dosing of medicament therefrom. The system is then reset. Other manual cutoffs/overrides may also be included.

In a subtle aspect of the present invention, it may be appreciated that the relevant timeframe for detecting, timing and alerting are determined by user action in relation to the system, and in particular by user action. The dose reminder capability is therefore independent of any particular defined external time zone (e.g. the local time zone relative to Greenwich Mean Time, as defined by the twenty four hour clock) because the user action defines its own ‘reminder timeframe’. This provides advantages over other known reminder systems, which are reliant on user reference to defined external time frames. The advantage is particularly great for the international traveller since complex calculations involving different local time zones are avoided.

It will be appreciated from the above description that the various components of the electronic dose reminder system interrelate with each other to provide the required functionality. The system may be configured in any suitable fashion using known electronic components and circuitry methods.

Suitably, the device additionally comprises an actuation detector for detecting actuation of any one of the medicament dispensers thereof wherein said actuation detector transmits actuation data to the electronic data management system.

The device may additionally comprise a safety mechanism to prevent unintended multiple actuations of the component medicament dispensers. The patient is thereby, for example, protected from inadvertently receiving multiple doses of medicament in a situation where they take a number of short rapid breaths. More preferably, the safety mechanism imposes a time delay between successive actuations of the release means. The time delay is typically of the order of from three to thirty seconds.

Suitably, the device additionally comprises a release detector for detecting release of medicament from the cassette, wherein said release detector transmits release data to the electronic data management system.

Suitably, the device additionally comprises a shake detector for detecting shaking of the medicament container (e.g. prior to actuation of the dispensing mechanism), wherein said shake detector transmits shake data to the electronic data management system.

Suitably, any actuation detector, release detector, or shake detector comprises a sensor for detecting any suitable parameter such as movement. Any suitable sensors are envisaged including the use of optical sensors. The release detector may sense any parameter affected by release of the medicament such as pressure, temperature, sound, moisture, carbon dioxide concentration and oxygen concentration.

Suitably, the medicament dispenser is actuable in response to the inward breath of a patient and includes a breath sensor of any suitable type (e.g. mechanical or electronic) for detecting that inward breath wherein optionally, the sensor communicates with an electronic control system. Thus, for example, in use the patient breathes in through the dispenser (e.g. through the mouthpiece); the breath is detected by the breath sensor; the sensor communicates with the electronic control system to convey an ‘inward breath detected’ signal; and the electronic control system responds by releasing medicament from one or more of the medicament containers for inhalation by the patient.

Suitably, the device additionally comprises a breath trigger for triggering one or all of the component medicament dispensers, said breath trigger being actuable in response to a trigger signal from the electronic data management system. Preferably, the electronic data management system includes a predictive algorithm or look-up table for deriving from the breath data when to transmit the trigger signal. For example, a real-time analysis of the patient breath waveform may be made and the trigger point derived by reference to that analysed waveform.

Suitably, the electronic data management system includes a predictive algorithm or look-up table for calculating the optimum amount of medicament to dispense.

Suitably, the memory on the electronic data management system includes a dose memory for storing dosage data and reference is made to the dose memory in calculating the optimum amount of medicament to dispense.

Suitably, the device additionally comprises a selector for selecting the amount of medicament to dispense from said dispensing mechanism. In one aspect, the selector is manually operable. In another aspect, the selector is operable in response to a signal from the transmitter on the electronic data management system.

Suitably, the device comprises in association with a body or housing thereof, a first transceiver for transmitting and receiving data and in association with the medicament container, a second transceiver for transmitting and receiving data, wherein data is transferable in two-way fashion from the first transceiver to the second transceiver. The data is preferably in digital form and suitable for transfer by electronic or optical means. A medicament dispenser of this general type is described in pending UK Patent Application No. 0020538.5.

One advantage of embodiments of this type is the ability to store many types of information in different parts of the memory structure of the transceivers. The information is furthermore stored in a form which is readily and accurately transferable. The information could for example, include manufacturing and distribution compliance information written to the memory at various points in the manufacturing or distribution process, thereby providing a detailed and readily accessible product history of the dispenser. Such product history information may, for example, be referred to in the event of a product recall. The compliance information could, for example, include date and time stamps. The information could also include a unique serial number stored in encrypted form or in a password protectable part of the memory which uniquely identifies the product and therefore may assist in the detection and prevention of counterfeiting. The information could also include basic product information such as the nature of the medicament and dosing information, customer information such as the name of the intended customer, and distribution information such as the intended product destination.

On loading or reloading the device with a medicament dispenser or ‘refill’ the second transceiver may, for example, read the unique serial number, batch code and expiry date of the medicament and any other information on the second transceiver. In this way the nature and concentration of the medicament, together with the number of doses used or remaining within the cassette, may be determined. This information can be displayed to the patient on a visual display unit. Other information, such as the number of times the medicament dispenser has been reloaded with a cassette, may also be displayed.

Similarly, should the cassette be removed from the holder before the supply of medicament is exhausted, the same data can be read from the second transceiver and the number of doses remaining or used determined. Other information, such as the date and time of administration of the drug, or environmental exposure data such as the minimum/maximum temperatures or levels of humidity the cassette has been exposed to, may also be read and displayed to the user.

In the event that the supply of medicament within any medicament container becomes exhausted, or that the shelf life of the medicament has expired, or that the first transceiver does not recognise the batch code on the second transceiver, activation of the dispenser may be prevented to safeguard the user. Activation may also be prevented if the medicament has been exposed to extreme environmental conditions for periods outwith the manufacturer's guidelines.

Data may be transferred to and from any transceiver during the period of use of the medicament dispenser by the patient. For example, the medicament dispenser may include an electronic data management system having various sensors associated therewith. Any data collected by the sensors or from any data collection system associated with the electronic data management system including a clock or other date/time recorder is transferable.

Data may be transferred each time the patient uses the device. Or alternatively, data may be stored in a database memory of the electronic data management system and periodically downloaded to any transceiver. In either case, a history of the usage of the device may be built up in the memory of a transceiver.

In one embodiment herein, a history of the usage of the device is transferred to the second transceiver. When the medicament carriers in the cassette are exhausted it is exchanged by the patient for a new refill cassette. At the point of exchange, which will typically occur at the pharmacy, data may be transferred from the exhausted cassette to the refill and vice-versa. Additionally, usage history data may be read from the refill and transferred to a healthcare data management system for example comprising a network computer system under the control of a healthcare data manager.

Methods are envisaged herein whereby the patient is given some sort of reward for returning the refill and making available the data comprised within the second transceiver. Methods are also envisaged herein whereby the healthcare data manager is charged for either receipt of the data from the second transceiver or for its use for commercial purposes. Any rewards or charging may be arranged electronically. The methods may be enabled by distributed or web-based computer network systems in which any collected data is accessible through a hub on the network. The hub may incorporate various security features to ensure patient confidentiality and to allow selective access to information collected dependent upon level of authorisation. The level of user authorisation may be allocated primarily to safeguard patient confidentiality. Beyond this the level of user authorisation may also be allocated on commercial terms with for example broader access to the database being authorised in return for larger commercial payments.

Suitably, the first and second transceiver each comprise an antenna or equivalent for transmitting or receiving data and connecting thereto a memory. The memory will typically comprise an integrated circuit chip. Either transceiver may be configured to have a memory structure which allows for large amounts of information to be stored thereon. The memory structure can be arranged such that parts of the memory are read-only, being programmed during/after manufacture, other parts are read/write and further parts are password protectable. Initial transfer of information (e.g. on manufacture or one dispensing) to or from any transceiver can be arranged to be readily achievable by the use of a reader which is remote from the medicament dispenser, thereby minimising the need for direct product handling. In further aspects, the reader can be arranged to simultaneously read or write to the memory of multiple transceivers on multiple medicament dispensers.

A suitable power source such as a battery, clockwork energy store, solar cell, fuel cell or kinetics-driven cell will be provided as required to any electronic component herein. The power source may be arranged to be rechargeable or reloadable.

Suitably, data is transferable in two-way fashion between the first and second transceiver without the need for direct physical contact therebetween. Preferably, data is transferable wirelessly between the first and second transceiver.

Suitably, the first transceiver is an active transceiver and the second transceiver is a passive transceiver. The term active is used to mean directly-powered and the term passive is used to mean indirectly-powered.

Suitably, the second transceiver comprises a label or tag comprising an antenna for transmitting or receiving energy; and an integrated circuit chip connecting with said antenna, and the first transceiver comprises a reader for said label or tag. In this case the label or tag is a passive transceiver and the reader is an active transceiver. Preferably, the reader will not need to be in direct contact with the tag or label to enable the tag or label to be read.

The tag may be used in combination and/or integrated with other traditional product labelling methods including visual text, machine-readable text, bar codes and dot codes.

Suitably, the integrated circuit chip has a read only memory area, a write only memory area, a read/write memory area or combinations thereof.

Suitably, the integrated circuit chip has a one-time programmable memory area. More preferably, the one-time programmable memory area contains a unique serial number.

Suitably, the integrated circuit chip has a preset memory area containing a factory preset, non-changeable, unique data item. The preset memory item is most preferably in encrypted form.

Suitably, the integrated circuit chip has plural memory areas thereon. Suitably, any memory area is password protected.

Suitably, any memory area contains data in encrypted form. Electronic methods of checking identity, error detection and data transfer may also be employed.

In one aspect, the integrated circuit has plural memory areas thereon including a read only memory area containing a unique serial number, which may for example be embedded at the time of manufacture; a read/write memory area which can be made read only once information has been written thereto; and a password protected memory area containing data in encrypted form which data may be of anti-counterfeiting utility.

Suitably, the tag is on a carrier and the carrier is mountable on the body or holder of the medicament dispenser or on the cassette.

In one aspect, the carrier is a flexible label. In another aspect, the carrier is a rigid disc. In a further aspect, the carrier is a rectangular block. In a further aspect, the carrier is a collar ring suitable for mounting to the neck of an aerosol container. Other shapes of carrier are also envisaged.

Suitably, the carrier is mouldable or weldable to the cassette or housing. Suitably, the carrier encases the tag. More preferably, the carrier forms a hermetic seal for the tag.

In one aspect, the carrier comprises an insulating material such as a glass material or, a paper material or an organic polymeric material such as polypropylene. Alternatively, the carrier comprises a ferrite material.

The energy may be in any suitable form including ultrasonic, infrared, radiofrequency, magnetic, optical and laser form. Any suitable channels may be used to channel the energy including fibre optic channels.

In one aspect, the second transceiver comprises a radiofrequency identifier comprising an antenna for transmitting or receiving radiofrequency energy; and an integrated circuit chip connecting with said antenna, and the first transceiver comprises a reader for said radiofrequency identifier. In this case the radiofrequency identifier is a passive transceiver and the reader is an active transceiver. An advantage of radiofrequency identifier technology is that the reader need not be in direct contact with the radiofrequency identifier tag or label to be read.

The radiofrequency identifier can be any known radiofrequency identifier. Such identifiers are sometimes known as radiofrequency transponders or radiofrequency identification (RFID) tags or labels. Suitable radiofrequency identifiers include those sold by Phillips Semiconductors of the Netherlands under the trade marks Hitag and Icode, those sold by Amtech Systems Corporation of the United States of America under the trade mark Intellitag, and those sold by Texas Instruments of the United States of America under the trade mark Tagit.

Suitably, the antenna of the RFID tag is capable of transmitting or receiving radiofrequency energy having a frequency of from 100 kHz to 2.5 GHz. Preferred operating frequencies are selected from 125 kHz, 13.56 MHz and 2.4 GHz.

In one aspect, the second transceiver comprises a magnetic label or tag comprising an antenna for transmitting or receiving magnetic field energy; and an integrated circuit chip connecting with said antenna, and the first transceiver comprises a reader for said magnetic label or tag. In this case the magnetic label or tag is a passive transceiver and the reader is an active transceiver.

A suitable magnetic label or tag comprises plural magnetic elements in mutual association whereby the magnetic elements move relative to each other in response to an interrogating magnetic field. A magnetic label or tag of this type is described in U.S. Pat. No. 4,940,966. Another suitable magnetic label or tag comprises a magnetorestrictive element which is readable by application of an interrogating alternating magnetic field in the presence of a magnetic bias field which results in resonance of the magnetorestrictive elements at different predetermined frequencies. A magnetic label of this type is described in PCT Patent Application No. WO92/12402. Another suitable magnetic label or tag comprising plural discrete magnetically active regions in a linear array is described in PCT Patent Application No. WO96/31790. Suitable magnetic labels and tags include those making use of Programmable Magnetic Resonance (PMR) (trade name) technology.

In another aspect, the second transceiver comprises a microelectronic memory chip and the first transceiver comprises a reader for said microelectronic memory chip. The microelectronic memory chip may comprise an Electrically Erasable Programmable Read Only Memory (EEPROM) chip or a SIM card-type memory chip. In this case the microelectronic memory chip is a passive transceiver and the reader is an active transceiver.

Any transceiver herein, particularly a passive transceiver may be mounted on or encased within any suitable inert carrier. The carrier may comprise a flexible sheet which may in embodiments be capable of receiving printed text thereon.

In one aspect, the first transceiver is integral with the body such that a single unit is comprised. The first transceiver may for example be encased within or moulded to the body.

In another aspect, the first transceiver forms part of a base unit which is reversibly associable with the body. The base unit may for example, form a module receivable by the body such as a snap-in module.

Suitably, the device additionally comprises a communicator for wireless communication with a network computer system to enable transfer of data between the network computer system and the electronic data management system. Dispensers employing such communicators are described in pending PCT Applications No.s PCT/EP00/09291 (PG3786), PCT/EP00/09293 (PG4029) and PCT/EP00/09292 (PG4159). Preferably, the communicator enables two-way transfer of data between the network computer system and the electronic data management system.

Suitably, the data is communicable between the network computer system and the electronic data management system in encrypted form. All suitable methods of encryption or partial encryption are envisaged. Password protection may also be employed. Suitably, the communicator employs radiofrequency or optical signals.

In one aspect, the communicator communicates via a gateway to the network computer system. In another aspect, the communicator includes a network server (e.g. a web server) such that it may directly communicate with the network.

In a further aspect, the communicator communicates with the gateway via a second communications device. Preferably, the second communications device is a telecommunications device, more preferably a cellular phone or pager. Preferably, the communicator communicates with the second communications device using spread spectrum radiofrequency signals. A suitable spread spectrum protocol is the Bluetooth (trade mark) standard which employs rapid (e.g. 1600 times a second) hopping between plural frequencies (e.g. 79 different frequencies). The protocol may further employ multiple sending of data bits (e.g. sending in triplicate) to reduce interference.

In one aspect, the network computer system comprises a public access network computer system. The Internet is one suitable example of a public access network computer system, wherein the point of access thereto can be any suitable entrypoint including an entrypoint managed by an Internet service provider. The public access network computer system may also form part of a telecommunications system, which may itself be either a traditional copper wire system, a cellular system or an optical network.

In another aspect, the network computer system comprises a private access network computer system. The private access network system may for example, comprise an Intranet or Extranet which may for example, be maintained by a health service provider or medicament manufacturer. The network may for example include password protection; a firewall; and suitable encryption means.

Preferably, the communicator enables communication with a user-specific network address in the network computer system.

The user-specific network address may be selected from the group consisting of a web-site address, an e-mail address and a file transfer protocol address. Preferably, the user-specific network address is accessible to a remote information source such that information from said remote information source can be made available thereto. More preferably, information from the user-specific network address can be made available to the remote information source.

In one aspect, the remote information source is a medicament prescriber, for example a doctors practice. Information transferred from the medicament prescriber may thus, comprise changes to prescription details, automatic prescription updates or training information. Information transferred to the medicament prescriber may comprise compliance information, that is to say information relating to the patient's compliance with a set prescribing programme. Patient performance information relating for example, to patient-collected diagnostic data may also be transferred to the medicament prescriber. Where the dispenser is an inhaler for dispensing medicament for the relief of respiratory disorders examples of such diagnostic data would include breath cycle data or peak flow data.

In another aspect, the remote information source is a pharmacy. Information transferred from the pharmacy may thus, comprise information relating to the medicament product. Information sent to the pharmacy may thus include prescription requests which have been remotely pre-authorised by the medicament prescriber.

In a further aspect, the remote information source is an emergency assistance provider, for example a hospital accident and emergency service or an emergency helpline or switchboard. The information may thus, comprise a distress or emergency assist signal which requests emergency assistance.

In a further aspect, the remote information source is a manufacturer of medicament or medicament delivery systems. Information transferred to the system may thus, comprise product update information. The system may also be configured to feed information back to the manufacturer relating to system performance.

In a further aspect, the remote information source is a research establishment. In a clinical trial situation, information may thus be transferred relating to the trial protocol and information relating to patient compliance fed back to the research establishment.

In a further aspect, the remote information source is an environmental monitoring station. Information relating to weather, pollen counts and pollution levels may thus be made accessible to the system.

Suitably, the device additionally comprises a geographic positioning system such as a global positioning system or a system which relies on the use of multiple communications signals and a triangulation algorithm.

The constituent medicaments of the plural medicament dose portions suitably, in combination comprise a combination medicament product. Suitably the medicaments are selected from the group consisting of albuterol, salmeterol, fluticasone propionate and beclomethasone dipropionate and salts or solvates thereof. Preferably, the combination comprises salmeterol xinafoate and fluticasone propionate.

### BRIEF DESCRIPTION OF THE DRAWINGS

The invention will now be described with reference to the accompanying drawings in which:

FIGS. 1 to 4 show a prior art dispenser device suitable for adaptation in accord with the invention, FIG. 1 being an underplan view, FIG. 2 a section on line A-A in FIG. 1, FIG. 3 a section on line B-B in FIG. 1, and FIG. 4 an exploded view on a smaller scale;

FIGS. 4a to 4d show the dispenser device of FIGS. 1 to 4 in successive stages of operation;

FIG. 5 shows an elongate blister form medicament pack suitable for use with the dispenser device of FIG. 1;

FIG. 6a shows a perspective view of a unitary dispenser device herein comprising multi-dose dry powder inhaler (MDPI) and metered dose inhaler (MDI) dispenser types;

FIG. 6b shows a sectional view of a detail of the unitary dispenser device of FIG. 6a;

FIG. 7a shows a perspective view of a unitary dispenser device herein comprising liquid spray inhaler (LSI) and metered dose inhaler (MDI) dispenser types; and

FIG. 7b shows a cut-away perspective view of a detail of the unitary dispenser device of FIG. 7a.

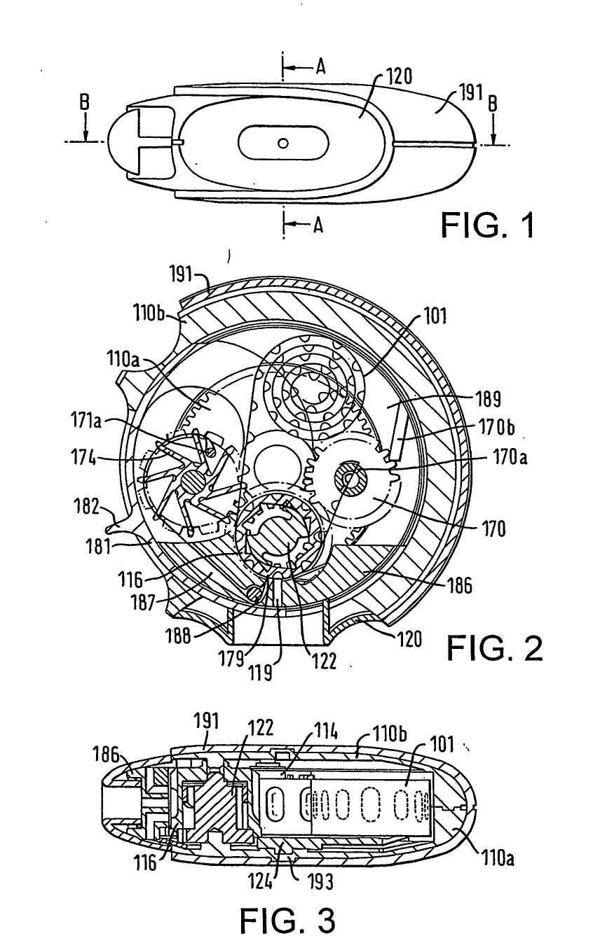

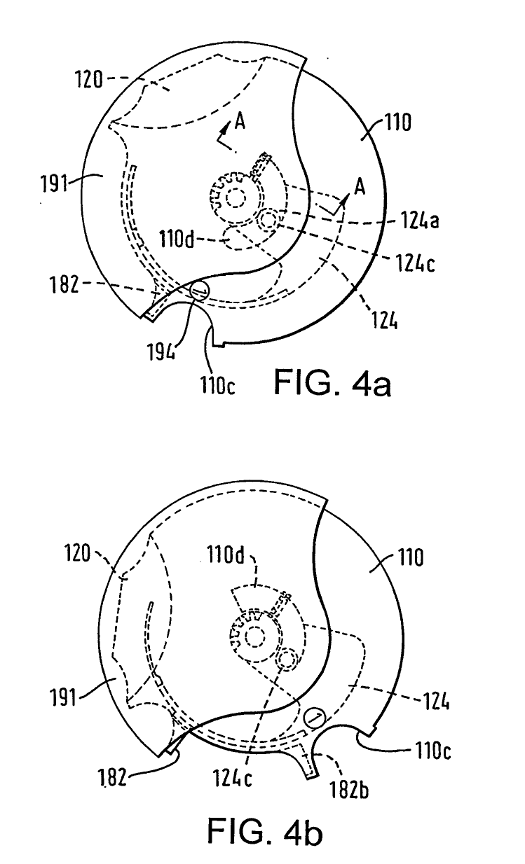

FIGS. 1 to 4 show a prior art dispenser device of the multi-dose dry powder inhaler (MDPI) type suitable for adaptation in accord with the invention. Suitable adaptation is described hereinafter. The dispenser device receives a flexible strip, here denoted as 101, comprising a base sheet 103 in which pockets 102 are defined and a lid sheet 104. The strip 101, is shown in more detail in FIG. 5. The lid sheet 104 has a loop 104a formed at the leading end thereof for engagement over a post 171a extending upwardly from a toothed wheel 171 (described below). The base sheet has a lead portion 103a of reduced width for engagement in a slot 170a formed in the base winding wheel 170 (described below). The leading end portions of the base sheet and lid sheet are not sealed together, as can be seen in FIG. 5.

The body 110 comprises a base 110a and a top 110b both of generally circular shape. When the device is assembled the base and top are snap-fitted together. The body defines a single internal chamber within which the strip 101 is housed and within which are also housed a wheel 114 for winding up the used portion of the lid sheet 104, a base winding wheel 170 and an index wheel 116. The index wheel 116 is hollow and an index ratchet wheel 122 is housed within it. All the wheels just mentioned are mounted in the chamber defined by the body, for rotational movement with respect thereto. A pawl 170b is attached to the body 110 and engages the teeth of the base winding wheel 170 to prevent the wheel moving anticlockwise, thus ensuring that the strip 101 can only proceed forwards through the device.

The lid winding wheel 114 is formed in two parts, namely a toothed wheel 171 having teeth 172 and a shaft 173, and a collapsible wheel 174 having a hollow central shaft 175 and a plurality of resilient arms 176, for example, as shown, eight such arms, extending from the central shaft 175 each at an angle to a radius. The toothed wheel 171 has a lug 177, which engages in a corresponding notch in the shaft 175 so that the wheels 171 and 174 rotate in unison.

The hollow index wheel 116 has external teeth 178, which mesh with the teeth of the base winding wheel 170 and the teeth of the wheel 171. Ratchet teeth 179 are formed on the internal walls of the index wheel 116, and the index ratchet wheel 122 has two pawls 180 which engage the ratchet teeth 179.

The device further comprises a lever 124 which comprises an arcuate wall 181 with a finger tab 182, and an arm 183 which extends inwardly from the wall 181 and carries an arcuate array of teeth 184 at its distal end. The lever is pivotally mounted to the centre of the base 110a for movement about an axis which is at the centre of the pitch circle of the teeth 184, the teeth 184 mesh with the teeth 185 on the index ratchet wheel 122.

A manifold 186 provides communication between the chamber within the body 110 and a mouthpiece 120. The manifold has a powder outlet 119 and also has a passageway 187 to allow used lid strip 104 to pass to the collapsible wheel 174. Optionally, a roller 188 may be provided to guide the strip 104 into the passageway 187.

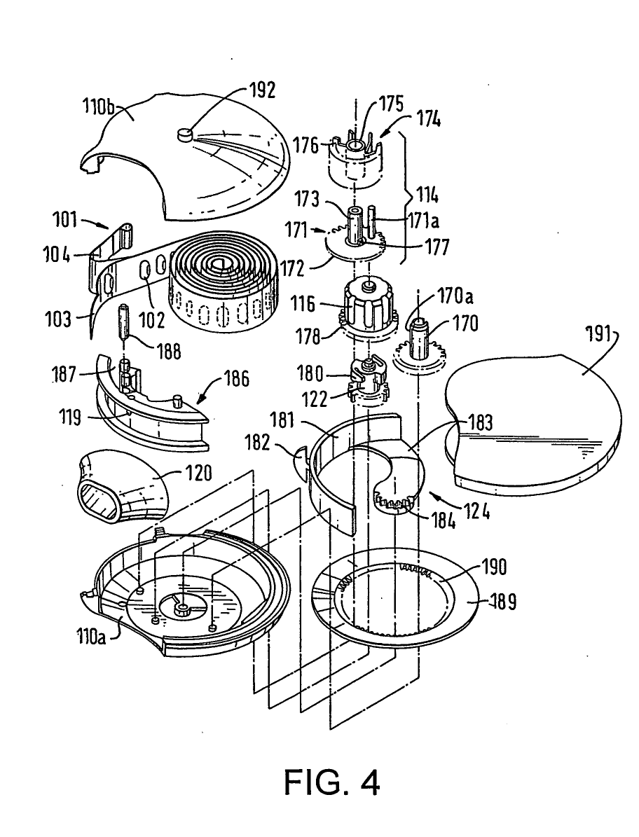

A dose monitor ring 189 having teeth 190 is arranged to be rotatable within the body base 110a. On its lower surface this bears indicia (not visible in the drawings) which can be viewed by the user through a window 194 in the body 110. It will be noted from FIGS. 4a to 4d that the window can be seen both when the cover 191 (see below) is closed and when it is open. The indicia indicate either exactly or approximately the number of doses left (or the number of doses used, if preferred). The ring 189 is rotated by virtue of the fact that its teeth 190 are engaged by the teeth 178 of the index wheel.

The device is provided under a cover 191 that is pivotally mounted on the body 110 by means of a lug 192 on the body top 110b and a corresponding lug 193 on the body base 110a. The cover is pivotal between an open position (shown in FIG. 2) in which the mouthpiece is exposed and a closed position in which it is not, as is described more fully below.

In operation, the user moves the cover 191 to its open position and then presses on the finger tab 182 of the lever 124 to cause it to move as the lever pivots. This makes the index ratchet wheel 122 rotate which, via the pawls 180, causes the index wheel 116 also to rotate. Rotation of the index wheel 116 produces rotation of both the base winding wheel 170 and the lid winding wheel 114, thus peeling the base sheet and lid sheet apart over a distance sufficient to expose a previously unopened pocket 102 opposite the end of the powder outlet 119 in the manifold. The patient can then inhale through the mouthpiece, as in the preceding embodiments.

Successive stages in the operation of the device are shown in FIGS. 4a to 4d. The device is in its closed position in FIG. 4a. The finger tab 182 of the lever 124 is at this stage in a recess 182b formed in the body 110 (seen more clearly in FIGS. 4b and 4c). The cover 119 is held stationary as the body 110 is rotated anticlockwise, a recess 110c being provided in the periphery of the body to enable the user to insert a finger for this purpose. The device is thus moved to the partly open position shown in FIG. 4b. During this process the lever 124 remains stationary with respect to the 191. This is achieved by the lever 124 being provided internally with a resilient arm 124a, the tip 124b of which engages in a recess 191a in the cover 191. The arm 124a is attached to the lever 124 via a cylindrical member 124c. As viewed in FIG. 4a, the arm 124a extends anticlockwise from the member 124c over an arc of about 90° degree. The cylindrical member 124c is guided in an arcuate slot 110d formed in the body 110. The slot 110d extends through an arc of about 180° degree, and in FIG. 4a the member 424c is shown as being approximately half way along its length. In FIG. 4b it is shown as being at one end.

The user continues to rotate the body 110 from the position shown in FIG. 4b to the position shown in FIG. 4c. During this further rotation tip 124b of the arm 124a jumps out of the recess 191a. This occurs because, with the member 124c at one end of the slot 10d, movement of the body 110 carries the member 124c with it in an anticlockwise direction and hence compels the arm 124a likewise to move anticlockwise. The user then moves the lever 124 by pushing on the finger tab 182 to cause it to rotate anticlockwise through the position shown in FIG. 4c to the position shown in FIG. 4d where the finger tab 182 re-enters the recess 182b. The steps thus far described both expose the mouthpiece 120 and open a fresh blister. The device is therefore now ready for the user to inhale.

After use, the body 110 is rotated clockwise, the lever 124 moving in unison with the body, to bring the device back to the position of FIG. 4a.

It will be noted that the collapsible wheel 174 in effect assumes the function of the clutch in the first embodiment. As more lid sheet is wound onto the wheel 174 the arms 176 gradually flex inwardly, and the effect is to keep the external diameter of the reel of wound up lid sheet substantially constant, while the internal diameter thereof gradually decreases.

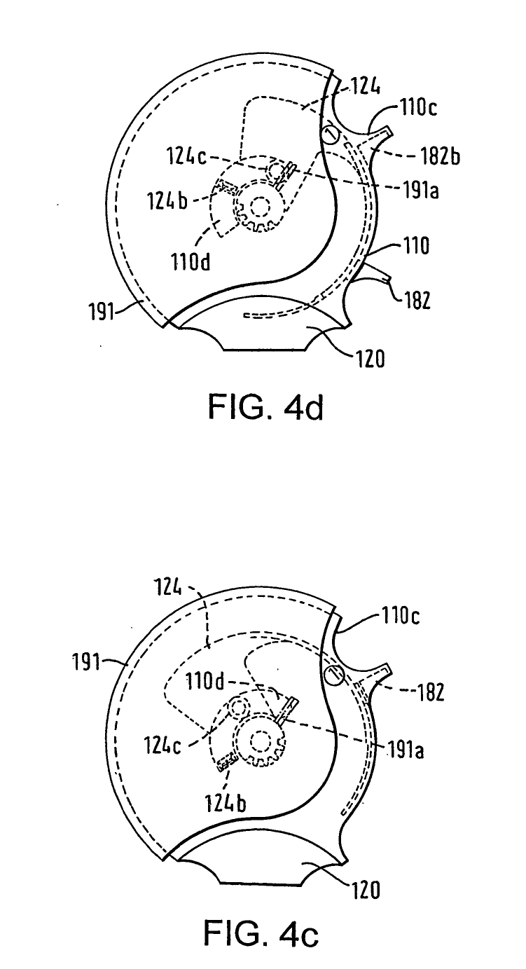

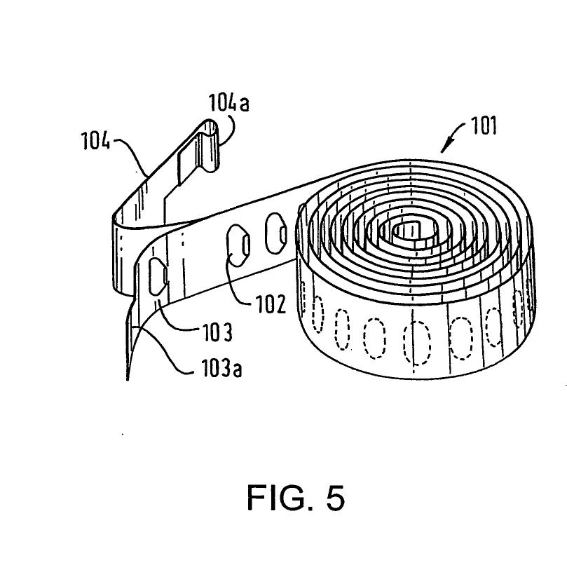

FIG. 6a shows a unitary dispenser device obtainable by suitable adaptation of the multi-dose dry powder inhaler (MDPI) dispenser device of FIGS. 1 to 5 to receive a metered dose inhaler (MDI) type dispenser. Components of the embodiment of FIG. 6, which correspond to those of the prior art dispenser of FIGS. 1 to 5 are denoted by the same reference numerals but with the addition of 100. FIG. 6b shows the interaction of the MDPI and MDI parts of the unitary dispenser in more detail.

The unitary dispenser device of FIG. 6a comprises a body 210 of generally circular shape. The body 210 defines a chamber within which a strip form blister pack (not visible in FIG. 6a) of the type shown in FIG. 5 is received. Individual medicament-containing blister pockets of the pack are accessible by strip advancement via an advancement and access mechanism as described in relation to the dispenser device of FIGS. 1 to 4. Thus, the strip is advanced in response to patient movement of lever 224 by finger action at finger tab 282, which results in opening of a blister 202 (as described in FIGS. 4a to 4b) in order that the medicament dose contained in the blister 202 may be inhaled through mouthpiece 220.

The body 210 is further provided with a rotationally mounted cover 291. In accord with the present invention, both the body 210 and the cover 291 are adapted to receive an aerosol canister 2100 having a metering valve dispenser 2102 of the type widely used in well-known metered dose inhaler (MDI) type medicament dispensers. The cover 291 is therefore provided with a cylindrical protrusion 2110 comprising a docking port 2112 sized and shaped to receive the neck 2104 of the aerosol canister 2100 such that when the canister 2100 is so-received the valve stem 2102 of the canister 2100 protrudes into the body 210 of the unitary device.

The receipt of the canister 2100 and in particular the valve stem 2102 within the body 210 of the unitary dispenser device may be better understood by reference to FIG. 6b. The valve stem 2102 is received within actuator block 2120, which defines a cylindrical valve stem-receiving channel 2122 having a stepped constriction 2124 provided therein and against which the tip 2106 of the valve stem 2102 seats when the stem 2102 is firmly inserted into the channel 2122. The block 2120 is also shaped to define an outlet 2126, which communicates with exit conduit 2130, which is arranged to guide the release of aerosol form medicament dose fired from the canister 2100 via the metering valve 2102.

Actuation of the MDI part of the unitary dispenser device occurs in relation to patient movement of the canister 2100 such that the tip 2106 of the valve stem 2102 seats hard against the step constriction 2124 of the receiving-channel 2122 of the stem block 2120. Further movement of the canister 2100 results in actuation of the valve 2102 (typically, a metering slide valve) thereby releasing a metered amount of aerosol form medicament into the actuator block 2120 from which it emerges at outlet 2126 to be guided into exit conduit 2130.

The relationship between the release of one component of the combination medicament product in aerosol form from the MDI canister 2100 part of the unitary dispenser and release of the complementary part of the combination medicament product in dry powder form from the MDPI dispenser part thereof is now described, again mainly with reference to FIG. 6b.

Referring to FIG. 6b, opened pocket 202 of blister strip 201 of the MDPI part of the unitary device may be seen to communicate with common outlet and mixing chamber 219 defined by manifold 286. Distal end 2132 of the exit conduit 2130 from the MDI part of the device may also be seen to feed into the common outlet and mixing chamber 219. In use, a patient may therefore inhale both aerosol form medicament (from the MDI part) and powder form medicament (from the MDPI part) through mouthpiece 220, which may be seen to communicate with the common outlet and mixing chamber 219.

Various modes of usage are envisaged. In one, less preferred, sequential usage mode a blister pocket 202 is first opened and the user inhales through the mouthpiece 220 to receive the powdered dose from that opened pocket. The user then actuates the valve 2102 of the MDI aerosol canister 2100 to release aerosol form medicament, which again is inhaled by the patient. Both dry powder and aerosol form medicaments are hence (sequentially) made available for inhalation.

In another, more preferred, usage mode a blister pocket 202 is first opened to bring the powdered medicament dose contents thereof into communication with the common outlet and mixing chamber 219. The user then actuates the valve 2102 of the MDI aerosol canister 2100 to release aerosol form medicament into the chamber 219. The kinetic energy comprised in the released aerosol form medicament is in part transferred to the powder contents of the blister pocket 202, which are also thereby aerosolised or at least agitated. The user inhales through the mouthpiece 220 (either contemporaneously with the firing of the MDI valve 2102 or soon thereafter) to receive both the released aerosol form medicament and the powdered dose from that opened pocket 202. Both dry powder and aerosol form medicament is hence (simultaneously) made available for inhalation by the user.

It will be appreciated that use of the unitary device of FIGS. 6a and 6b is usually configured such that a first component of a combination medicament product is contained in the MDI canister 2100 and a second component of the combination medicament component is contained with the blister pockets 202 of the MDPI part of the unitary dispenser. Simultaneous or sequential delivery of a ‘mixed form’ combination medicament product is thereby, enabled.

It may also be appreciated that any or all of the manual user actions described above may be suitably automated. Thus, in one aspect either or both of blister pocket 202 opening and firing of the MDI valve 2102 may be arranged to be responsive to the inward breath of the patient. Suitable timing delays between the various device actions may also be incorporated for example, by placing the breath monitoring, pocket opening and valve firing actions under the control of a suitable electronic control system.

In a variation of the unitary dispenser of FIGS. 6a and 6b, the MDI dispenser part is replaced by a liquid spray inhaler (LSI) type dispenser arranged to deliver a spray of liquid form droplets of suitable medicament formulation.

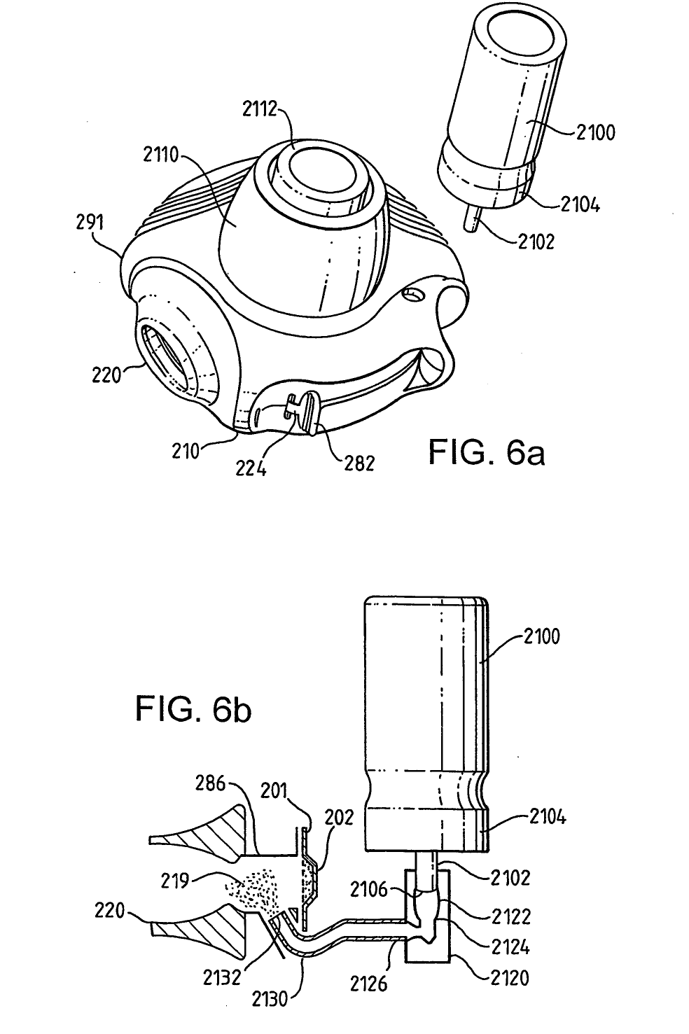

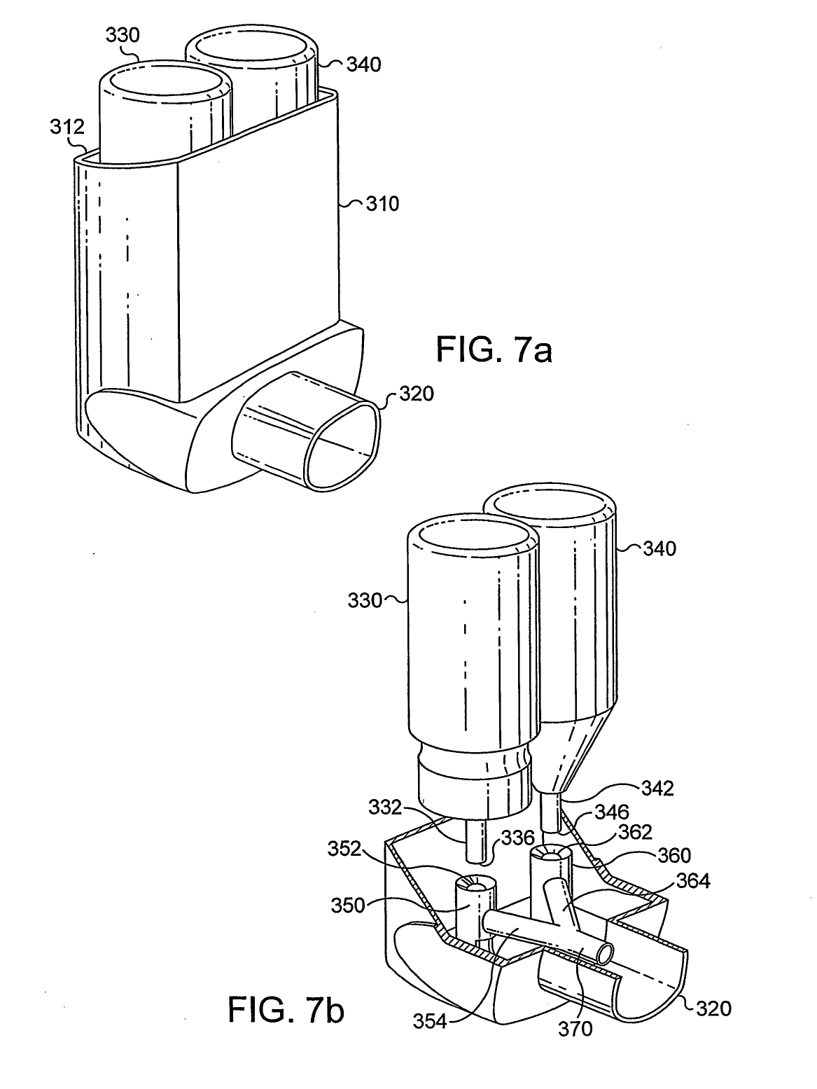

FIG. 7a shows a unitary dispenser device obtainable by suitable adaptation of well-known L-shaped (or ‘boot-shaped’) form metered dose inhaler (MDI) to incorporate a liquid spray inhaler (LSI) type dispenser. FIG. 7b shows the interaction of the MDI and LSI parts of the unitary dispenser in more detail.

The unitary dispenser device of FIG. 7a comprises a body 310 of L-shaped form. The body 310 defines an upwardly extending (as shown) protrusion defining a chamber 312 that is sized and shaped for side-by-side receipt of an MDI canister 330 and an LSI liquid pump container vial 340. The body 310 also defines a common mouthpiece 320 through which a user may inhale.

The receipt of the canister 330 and liquid container vial 340 within the body 310 of the unitary dispenser device may be better understood by reference to FIG. 7b. Valve stem 332 of the MDI canister is received within first actuator block 350, which defines a cylindrical valve stem-receiving channel 352 having a stepped constriction (not visible) provided therein and against which the tip 336 of the valve stem 332 seats when the stem 332 is firmly inserted into the channel 352. The first actuator block 350 is also shaped to define an outlet pipe 354, which communicates with common exit conduit 370, which is arranged to guide released medicament dose to the common mouthpiece 320. Pump outlet 342 of the LSI container 340 is similarly received within second actuator block 360, which defines a cylindrical pump outlet-receiving channel 362 having a stepped constriction (not visible) provided therein and against which the tip 346 of the pump outlet 342 seats when the pump outlet 342 is firmly inserted into the channel 362. The second actuator block 360 is also shaped to define an outlet pipe 364, which communicates with the common exit conduit 370.

Actuation of the MDI and LSI parts of the unitary dispenser device occurs respectively in response to patient-induced firing of the MDI part or pumped action at the LSI part.

In more detail, firing of the MDI part occurs in relation to patient movement of the canister 330 such that the tip 336 of the valve stem 332 seats hard against the step constriction (not visible) of the receiving-channel 352 of the first actuator block 350. Further movement of the canister 330 results in actuation of the valve 332 (typically, a metering slide valve) thereby releasing a metered amount of aerosol form medicament into the first actuator block 350 from which it emerges at outlet pipe 354 to be guided into the common exit conduit 370. Pumped spraying of the LSI part occurs in relation to patient movement of the liquid container vial 340 such that the tip 346 of the pump outlet 332 seats hard against the step constriction (not visible) of the receiving-channel 362 of the second actuator block 360. Further movement of the container 340 results in actuation of the pump 342 (typically, a metering pump) thereby releasing a metered amount of spray form medicament into the second actuator block 360 from which it emerges at outlet pipe 364 to be guided into the common exit conduit 370.

Various modes of usage are envisaged. In one, less preferred, sequential usage mode the MDI part and the LSI part of the unitary dispenser are actuated in sequential fashion (in either order). Both liquid spray and aerosol form medicaments are hence sequentially made available for inhalation by the user.

In another, more preferred, the MDI part is fired essentially simultaneously with pumped actuation of the LSI part. The user thus actuates the valve 332 of the MDI aerosol canister 330 to release aerosol form medicament into the common exit conduit 370 simultaneously with actuation of the LSI pump dispenser 342 to release liquid form medicament also into the common exit conduit 370. The user inhales through the mouthpiece 320 to receive both the released aerosol and liquid form medicaments for simultaneous inhalation.

It will be appreciated that use of the unitary device of FIGS. 7a and 7b is usually configured such that a first component of a combination medicament product is contained in the MDI canister 330 and a second component of the combination medicament component is contained within the container vial 340 of the LSI part of the unitary dispenser. Simultaneous or sequential delivery of a ‘mixed form’ combination medicament product is thereby, enabled.

It may also be appreciated that any or all of the manual user actions described above may be suitably automated. Thus, in one aspect either or both of firing of the MDI valve 332 and actuation of the pump 342 may be arranged to be responsive to the inward breath of the patient. Suitable timing delays between the various dispenser device actions may also be incorporated for example, by placing the breath monitoring, pump actuation and valve firing actions under the control of a suitable electronic control system.

In a variation of the unitary dispenser of FIGS. 7a and 7b, the liquid spray inhaler (LSI) type dispenser part is replaced by an alternative type of liquid spray inhaler (LSI) type dispenser in which a liquid spray is obtained by vibration of a suitably configured mesh to which liquid form medicament formulation is made available.

It may be appreciated that any of the parts of the device or any medicament thereof, which contacts medicament may be coated with materials such as fluoropolymer materials (e.g. PTFE or FEP) which reduce the tendency of medicament to adhere thereto. Any movable parts may also have coatings applied thereto which enhance their desired movement characteristics. Frictional coatings may therefore be applied to enhance frictional contact and lubricants (e.g. silicone oil) used to reduce frictional contact as necessary.

The device of the invention is suitable for dispensing differently formulated parts of medicament combination products, particularly for the treatment of respiratory disorders such as asthma and chronic obstructive pulmonary disease (COPD), bronchitis and chest infections.

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate (e.g. as the sodium salt), ketotifen or nedocromil (e.g. as the sodium salt); antiinfectives e.g., cephalosporins, penicillins, streptomycin, sulphonamides, tetracyclines and pentamidine; antihistamines, e.g., methapyrilene; anti-inflammatories, e.g., beclomethasone (e.g. as the dipropionate ester), fluticasone (e.g. as the propionate ester), flunisolide, budesonide, rofleponide, mometasone e.g. as the furoate ester), ciclesonide, triamcinolone (e.g. as the acetonide) or 6α,9α-difluoro-11β-hydroxy-16α-methyl-3-oxo-17α-propionyloxy-androsta-1,4-diene-17β-carbothioic acid S-(2-oxo-tetrahydro-furan-3-yl) ester; antitussives, e.g., noscapine; bronchodilators, e.g., albuterol (e.g. as free base or sulphate), salmeterol (e.g. as xinafoate), ephedrine, adrenaline, fenoterol (e.g. as hydrobromide), formoterol (e.g. as fumarate), isoprenaline, metaproterenol, phenylephrine, phenylpropanolamine, pirbuterol (e.g. as acetate), reproterol (e.g. as hydrochloride), rimiterol, terbutaline (e.g. as sulphate), isoetharine, tulobuterol or 4-hydroxy-7-[2-[[2-[[3-(2-phenylethoxy)propyl]sulfonyl]ethyl]amino]ethyl-2(3H)-benzothiazolone; adenosine 2a agonists, e.g. 2R,3R,4S,5R-[6-Amino-2-(1S-hydroxymethyl-2-phenyl-ethylamino)-purin-9-yl]-5-(2-ethyl-2H-tetrazol-5-yl)-tetrahydro-furan-3,4-diol (e.g. as maleate); α₄ integrin inhibitors e.g. (2S)-3-[4-({[4-(aminocarbonyl)-1-piperidinyl]carbonyl}oxy)phenyl]-2-[((2S)-4-methyl-2-{[2-(2-methylphenoxy) acetyl]amino}pentanoyl)amino]propanoic acid (e.g. as free acid or potassium salt), diuretics, e.g., amiloride; anticholinergics, e.g., ipratropium (e.g. as bromide), tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon; vaccines, diagnostics, and gene therapies. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Preferred components of combinations of active ingredients contain a bronchodilator in combination with an anti-inflammatory. The bronchodilator is suitably a beta-agonist, particularly a long-acting beta-agonist (LABA). Suitable bronchodilators include salbutamol (e.g., as the free base or the sulphate salt), salmeterol (e.g., as the xinafoate salt) and formoterol (eg as the fumarate salt). The anti-inflammatory is suitably an anti-inflammatory steroid. Suitably anti-inflammatory compounds include a beclomethasone ester (e.g., the dipropionate), a fluticasone ester (e.g., the propionate) or budesonide or any salt or solvate thereof. One preferred combination of components comprises fluticasone propionate and salmeterol, or any salt or solvate thereof (particularly the xinafoate salt). A further combination of components of particular interest is budesonide and formoterol or any salt or solvate thereof (e.g. formoterol as the fumarate salt).

Preferred components of combinations of active ingredients contain salbutamol (e.g., as the free base or the sulphate salt) or salmeterol (e.g., as the xinafoate salt) or formoterol (eg as the fumarate salt) in combination with an anti-inflammatory steroid such as a beclomethasone ester (e.g., the dipropionate) or a fluticasone ester (e.g., the propionate) or budesonide. A particularly preferred combination of components comprises fluticasone propionate and salmeterol, or a salt thereof (particularly the xinafoate salt). A further combination of components of particular interest is budesonide and formoterol (e.g. as the fumarate salt).

Generally, powdered medicament particles suitable for delivery to the bronchial or alveolar region of the lung have an aerodynamic diameter of less than 10 micrometers, preferably less than 6 micrometers. Other sized particles may be used if delivery to other portions of the respiratory tract is desired, such as the nasal cavity, mouth or throat. The medicament may be delivered as pure drug, but more appropriately, it is preferred that medicaments are delivered together with excipients (carriers) which are suitable for inhalation. Suitable excipients include organic excipients such as polysaccharides (i.e. starch, cellulose and the like), lactose, glucose, mannitol, amino acids, and maltodextrins, and inorganic excipients such as calcium carbonate or sodium chloride. Lactose is a preferred excipient.

Particles of powdered medicament and/or excipient may be produced by conventional techniques, for example by micronisation, milling or sieving. Additionally, medicament and/or excipient powders may be engineered with particular densities, size ranges, or characteristics. Particles may comprise active agents, surfactants, wall forming materials, or other components considered desirable by those of ordinary skill.

The excipient may be included with the medicament via well-known methods, such as by admixing, co-precipitating and the like. Blends of excipients and drugs are typically formulated to allow the precise metering and dispersion of the blend into doses. A standard blend, for example, contains 13000 micrograms lactose mixed with 50 micrograms drug, yielding an excipient to drug ratio of 260:1. Dosage blends with excipient to drug ratios of from 100:1 to 1:1 may be used. At very low ratios of excipient to drug, however, the drug dose reproducibility may become more variable.

Aerosol formulations suitable for use with metered dose inhaler (MDI) dispensers typically comprise a propellant. Suitable propellants include P11, P114 and P12, and the CFC-free hydrofluoroalkane propellants HFA-134a and HFA-227.

The MDI aerosol formulation may additionally contain a volatile adjuvant such as a saturated hydrocarbon for example propane, n-butane, isobutane, pentane and isopentane or a dialkyl ether for example dimethyl ether. In general, up to 50% w/w of the propellant may comprise a volatile hydrocarbon, for example 1 to 30% w/w. However, formulations, which are free or substantially free of volatile adjuvants are preferred. In certain cases, it may be desirable to include appropriate amounts of water, which can be advantageous in modifying the dielectric properties of the propellant.

A polar co-solvent such as C₂₋₆ aliphatic alcohols and polyols e.g. ethanol, isopropanol and propylene glycol, preferably ethanol, may be included in the MDI aerosol formulation in the desired amount to improve the dispersion of the formulation, either as the only excipient or in addition to other excipients such as surfactants. Suitably, the drug formulation may contain 0.01 to 30% w/w based on the propellant of a polar co-solvent e.g. ethanol, preferably 0.1 to 20% w/w e.g. about 0.1 to 15% w/w. In aspects herein, the solvent is added in sufficient quantities to solubilise the part or all of the medicament component, such formulations being commonly referred to as solution formulations.

A surfactant may also be employed in the MDI aerosol formulation. Examples of conventional surfactants are disclosed in EP-A-372,777. The amount of surfactant employed is desirable in the range 0.0001% to 50% weight to weight ratio relative to the medicament, in particular, 0.05 to 5% weight to weight ratio.

The final aerosol formulation desirably contains 0.005-10% w/w, preferably 0.005 to 5% w/w, especially 0.01 to 1.0% w/w, of medicament relative to the total weight of the formulation.

The device of the invention is in one aspect suitable for dispensing medicament for the treatment of respiratory disorders such as disorders of the lungs and bronchial tracts including asthma and chronic obstructive pulmonary disorder (COPD). In another aspect, the invention is suitable for dispensing medicament for the treatment of a condition requiring treatment by the systemic circulation of medicament, for example migraine, diabetes, pain relief e.g. inhaled morphine.

Accordingly, there is provided the use of a device according to the invention for the treatment of a respiratory disorder, such as asthma and COPD. Alternatively, the present invention provides a method of treating a respiratory disorder such as, for example, asthma and COPD, which comprises administration by inhalation of an effective amount of medicament product as herein described from a device of the present invention.

The amount of any particular medicament compound or a pharmaceutically acceptable salt, solvate or physiologically functional derivative thereof which is required to achieve a therapeutic effect will, of course, vary with the particular compound, the route of administration, the subject under treatment, and the particular disorder or disease being treated. The medicaments for treatment of respiratory disorders herein may for example, be administered by inhalation at a dose of from 0.0005 mg to 10 mg, preferably 0.005 mg to 0.5 mg. The dose range for adult humans is generally from 0.0005 mg to 100 mg per day and preferably 0.01 mg to 1 mg per day.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. A unitary medicament dispenser device for use in the delivery of a first medicament and at least one further medicament as a combination medicament product, the device comprising
a first medicament dispenser for the delivery of said first medicament; and
at least one further medicament dispenser for the delivery of said at least one further medicament,
wherein said first medicament dispenser and said at least one further medicament dispenser enable the first and the at least one further medicament to be kept separate until the point of delivery, and the first medicament dispenser is different in type to the at least one further medicament dispenser.

2. A medicament dispenser device according to claim 1, wherein the device comprises the first medicament dispenser and only one further medicament dispenser.

3. A medicament dispenser device for use in the inhaled delivery of the first medicament and at least one further medicament, in which the first medicament dispenser is selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI) and the at least one further medicament dispenser is selected from the group consisting of a reservoir dry powder inhaler (RDPI), a multi-dose dry powder inhaler (MDPI), a unit dose dry powder inhaler (UDPI), a metered dose inhaler (MDI) and a liquid spray inhaler (LSI).

4. A medicament dispenser device according to claim 1, wherein, the first medicament dispenser is a reservoir dry powder inhaler (RDPI), and the at least one further medicament dispenser is of a type selected from the group consisting of a multi-dose dry powder inhaler (MDPI), a metered dose inhaler (MDI) and a liquid spray inhaler (LSI).

5. A medicament dispenser device according to claim 1 wherein, the first medicament dispenser is a multi-dose dry powder inhaler (MDPI), and the at least one further medicament dispenser is of a type selected from the group consisting of a reservoir dry powder inhaler (RDPI), a metered dose inhaler (MDI) and a liquid spray inhaler (LSI).

6. A medicament dispenser device according to claim 1 wherein the first medicament dispenser is a metered dose inhaler (MDI), and the at least one further medicament dispenser is of a type selected from the group consisting of a reservoir dry powder inhaler (RDPI), a multi-dose dry powder inhaler (MDPI) and a liquid spray inhaler (LSI).

7. A medicament dispenser device according to claim 1 wherein the first medicament dispenser is a liquid spray inhaler (LSI), and the at least one further medicament dispenser is of a type selected from the group consisting of a reservoir dry powder inhaler (RDPI), a multi-dose dry powder inhaler (MDPI) and a metered dose inhaler (MDI).

8. A medicament dispenser device according to claim 3, wherein the multi-dose dry powder inhaler (MDPI) is arranged to reversibly receive a multi-dose blister pack suitable for containing medicament in dry powder form.

9. A medicament dispenser device according to claim 8, wherein the multi-dose blister pack comprises plural blisters arranged sequentially along an elongate strip.

10. A medicament dispenser device according to claim 9, wherein the strip form multi-dose blister pack comprises an elongate base sheet having plural blister pockets defined therein; and secured thereto and elongate lid sheet, wherein said elongate base sheet and lid sheet are peelably separable to enable access to said blister pockets.

11. A medicament dispenser device according to claim 3, wherein the metered dose inhaler (MDI) is arranged to reversibly receive an aerosol canister suitable for containing medicament in dry powder form.

12. A medicament dispenser device according to claim 3, wherein the metered dose inhaler (MDI) is arranged to reversibly receive a liquid container suitable for containing medicament in liquid form.

13. A medicament dispenser device according to claim 1, additionally comprising a coupled actuator for the first medicament dispenser and the at least one further medicament dispenser.

14. A medicament dispenser according to claim 1, wherein device additionally comprises a mixing chamber including inlets for receiving medicament from the first and at least one further medicament dispenser and an outlet for delivery of combination medicament product to the patient for inhalation.

15. A medicament dispenser device according to claim 14, wherein said outlet communicates with a common mouthpiece.

16. A medicament dispenser device according to claim 1, additionally comprising a breath sensor for sensing the breath of a patient wherein actuation of the first medicament dispenser and/or the at least one further medicament dispenser is responsive to said breath sensor.

17. A medicament dispenser device according to claim 1, wherein the first medicament dispenser includes a medicament container for containing the first medicament and the at least one further medicament dispenser includes at least one further medicament container for containing the at least one further medicament.

18. A medicament dispenser device according to claim 17, wherein the first medicament container contains the first medicament and the at least one further medicament container contains at least one further medicament.

19. A medicament dispenser device according to claim 18, wherein the first medicament comprises a bronchodilator and the at least one further medicament comprises an anti-inflammatory.

20. A medicament dispenser device according to claim 19, wherein said bronchodilator is a beta-agonist and said anti-inflammatory is a steroid.

21. A medicament dispenser device according to claim 20, wherein said bronchodilator is selected from the group consisting of salbutamol, salmeterol, formoterol and any salts or solvates thereof and mixtures thereof.

22. A medicament dispenser device according to claim 20, wherein said anti-inflammatory is selected from the group consisting of a beclomethasone ester, fluticasone ester, budesonide and any salt or solvates thereof and mixtures thereof.

23. A method of delivering a combination medicament product comprising the steps of
a.) providing a patient in need thereof a medicament dispenser device according to claim 1 and
b.) dispensing a combination medicament product from said device.
